(19) United States
(12) Patent Application Publication
(10) Pub. No.: US 2009/0151089 A1
(43) Pub. Date: Jun. 18, 2009

(54) COMPOSITION FOR THE OXIDATION DYEING OF KERATIN FIBERS COMPRISING AT LEAST ONE NONIONIC DERIVATIVE OF CELLULOSE WITH AT LEAST ONE HYDROPHOBIC SUBSTITUENT, AT LEAST ONE FATTY ACID ESTER, AND AT LEAST ONE OXIDATION DYE, AND OXIDATION DYEING PROCESS THEREFOR

(76) Inventor: Marie-Pascale AUDOUSSET, Asnieres (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/267,800

(22) Filed: Nov. 10, 2008

Related U.S. Application Data
(60) Provisional application No. 60/996,698, filed on Nov. 30, 2007.

(30) Foreign Application Priority Data
Nov. 9, 2007 (FR) 0758913

(51) Int. Cl. A61K 8/41; A61Q 5/10

(52) U.S. Cl. 8/408; 8/406; 8/421

(57) ABSTRACT

The present disclosure relates to a dye composition for keratin fibers, for example the human keratin fibers such as the hair, comprising, in a medium suitable for dyeing: at least one nonionic derivative of cellulose comprising at least one hydrophobic substituent containing from 8 to 30 carbon atoms; at least one C₈-C₃₀ fatty acid ester; and at least one oxidation dye. The present disclosure also relates to a process for dyeing keratin fibers using such a composition.

This application claims benefit of U.S. Provisional Application No. 60/996,698, filed Nov. 30, 2007, the contents of which are incorporated herein by reference. This application also claims benefit of priority under 35 U.S.C. § 119 to French Patent Application No. FR 0758913, filed Nov. 9, 2007, the contents of which are also incorporated herein by reference.

The present disclosure relates to a composition for the oxidation dyeing of keratin fibers, for instance, human keratin fibers such as the hair, comprising at least one nonionic derivative of cellulose modified with at least one hydrophobic group, at least one fatty acid ester, and at least one oxidation dye.

The present disclosure also relates to the use of this composition for dyeing keratin fibers and also to the dyeing process using this composition.

It is known to dye keratin fibers such as human hair with dye compositions comprising oxidation dye precursors, generally known as oxidation bases, such as ortho- or para-phenylenediamines, ortho- or para-aminophenols and heterocyclic compounds. These oxidation bases are colorless or weakly colored compounds which, in combination with oxidizing products, can give rise, via an oxidative condensation process, to colored compounds.

It is also known that it is possible to vary the shades obtained with these oxidation bases by combining them with couplers or coloring modifiers, the latter being chosen from, for example, aromatic meta-diamines, meta-aminophenols, meta-diphenols and certain heterocyclic compounds such as indole compounds.

The variety of the molecules involved as oxidation bases and couplers makes it possible to obtain a rich palette of colors.

It is desired that the “permanent” coloring obtained by virtue of these oxidation dyes should, moreover, meet a certain number of requirements.

For instance, it should have no toxicological drawbacks, it should allow shades to be obtained in the desired strength, and it should show good color-fastness with respect to external agents such as light, bad weather, washing, permanent-waving, perspiration and rubbing.

The dyes should also allow white hair to be covered and, finally, should be as nonselective as possible, i.e. they should make it possible to obtain the smallest possible differences in coloring along the same keratin fiber, which is generally differently sensitized (i.e. damaged) between its tip and its root.

Moreover, the compositions obtained should, in addition, have good rheological properties, while at the same time conserving good coloring properties. For instance, these compositions should not run on the face or out of the areas intended to be dyed, when they are applied, such as after mixing with an oxidizing agent.

Improving the power of dyeing by combining a para-phenylenediamine oxidation base and at least one nonionic amphiphilic polymer such as hydroxycellulose modified with a hydrophobic group is discussed in International Patent Application Publication No. WO 98/03150.

However, these compositions do not entirely meet the abovementioned requirements and can be improved, for instance, in terms of dyeing properties, such as in terms of dyeing selectivity and power. Thus, there is a need in the art to obtain stable hair dyeing compositions, for example, in the form of creams, which are easy to prepare and to apply, which may contain high concentrations of dyes in the form of salts, which have good rheological qualities and which produce strong, relatively nonselective colorations that withstand the various attacks that keratin fibers may be subjected to.

Accordingly, one aspect of the present disclosure, is a dye composition for keratin fibers, for example, human keratin fibers such as the hair, comprising, in a medium suitable for dyeing:

(A) at least one nonionic derivative of cellulose comprising at least one hydrophobic substituent containing from 8 to 30 carbon atoms;

(B) at least one C₈-C₃₀ fatty acid ester; and

(C) at least one oxidation dye.

The dye compositions according to the present disclosure may have at least one of the following properties:
they make it possible to obtain compositions with a viscosity corresponding to a cream, which are stable over time,
they stand out by virtue of the fact that they could be easily mixed with the oxidizing composition,
they stand out by virtue of the rheological qualities of the creams obtained (good viscosity of cream as a mixture),
they are easy to apply after mixing with the oxidizing composition at the time the dyeing is carried out (qualities of use on the head).

In addition, the compositions according to the present disclosure may make it possible to obtain compositions capable of producing colorings with varied, chromatic, powerful, aesthetic and relatively nonselective shades which are uniform over all the keratin fibers, for example, human keratin fibers such as the hair, and which are highly resistant to the various attacks to which the fibers may be subjected.

Another aspect of the present disclosure comprises a process for dyeing keratin fibers, wherein the cosmetic composition according to the present disclosure is used.

Another aspect of the present disclosure relates to the use of this cosmetic composition for dyeing keratin fibers, for example, human keratin fibers such as the hair.

Other features, aspects, subjects and benefits of the present disclosure will emerge more clearly on reading the description and the non-limiting examples which follow.

Unless otherwise indicated, the limits of the ranges of values which are given in the context of the present disclosure are included in these ranges.

As used herein, “derivative of cellulose” means at least one compound comprising at least one cellobiose unit having the structure:

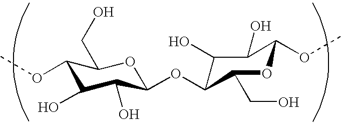

wherein at least one hydroxyl group may be substituted.

The at least one nonionic derivative of cellulose comprising at least one hydrophobic substituent (A) in accordance with the present disclosure is chosen from amphiphilic polymers that are associative in nature. The at least one nonionic derivative of cellulose comprising at least one hydrophobic substituent (A) comprises hydrophilic units and hydrophobic units and are capable of interacting and of associating with one another or with other molecules, reversibly, for example, by the presence of their hydrophobic chains.

In at least one embodiment of the present disclosure, the at least one cellulose derivative is a cellulose ether comprising at least one hydrophobic substituent containing from 8 to 30 carbon atoms.

The at least one nonionic derivative of cellulose comprising at least one hydrophobic substituent in accordance with the present disclosure can be prepared from water-soluble nonionic ethers of cellulose, wherein all or some of the reactive hydroxyl functional groups are substituted with at least one hydrophobic chain containing from 8 to 30 carbon atoms, for example, from 10 to 22 carbon atoms, such as 16 carbon atoms. The reaction steps involved in the preparation of the cellulose derivatives of the disclosure are known to those skilled in the art.

The nonionic ethers of cellulose chosen for preparing the at least one nonionic derivative of cellulose comprising at least one hydrophobic substituent according to the disclosure may have a degree of nonionic substitution, for example, methyl, hydroxyethyl or hydroxypropyl group(s), that is sufficient to be water-soluble, i.e. to form a substantially clear solution when dissolved in water at 25° C. at the concentration of 1% by weight.

The nonionic ethers of cellulose chosen for preparing the at least one nonionic derivatives of cellulose comprising at least one hydrophobic substituent according to the disclosure may have a relatively low number-average molar mass, of less than 800,000 g/mol, for example, ranging from 50,000 to 700,000 g/mol, or as another example, ranging from 200,000 to 600,000 g/mol.

According to at least one embodiment, the at least one cellulose derivative of the disclosure is chosen from hydroxyethylcelluloses comprising at least one hydrophobic substituent containing from 8 to 30 carbon atoms.

The at least one nonionic derivative of cellulose used according to the disclosure is substituted with at least one entity chosen from aliphatic and aromatic, saturated and unsaturated, linear, branched and cyclic C₈-C₃₀ hydrocarbon chains, that may be attached to the cellulose ether substrate via an ether, ester or urethane bond, such as an ether bond.

According to at least one embodiment, the at least one hydrophobic substituent used as substituents of the at least one nonionic derivatives of cellulose according to the present disclosure may be chosen from C₈-C₃₀ alkyl, arylalkyl and alkylaryl groups, such as C₁₀-C₂₂ alkyl, arylalkyl and alkylaryl groups.

For example, In at least one embodiment, the at least one hydrophobic substituent according to the present disclosure is chosen from a saturated alkyl chains.

According to another embodiment, the at least one hydrophobic substituent according to the present disclosure is chosen from cetyl groups.

The at least one nonionic derivative of cellulose with at least one hydrophobic substituent according to the present disclosure has a viscosity, for instance, ranging from 100 to 100,000 mPa·s, such as ranging from 200 to 20,000 mPa·s, measured at 25° C. in a solution at 1% by weight of polymer in water, this viscosity being determined conventionally using a Brookfield LVT viscometer at 6 rpm with the No. 3 spindle.

The degree of hydrophobic substitution of the at least one hydrophilic nonionic derivative of cellulose used according to the present disclosure can, for example, range from 0.1% to 10% by weight, such as, from 0.1% to 1% by weight, and further as another example, from 0.4% to 0.8% by weight, of the total weight of the polymer.

Among the nonionic derivatives of cellulose with at least one hydrophobic substituent that can be used in the compositions described herein include, but are not limited to, the cetyl hydroxyethylcelluloses sold under the names NATROSOL PLUS GRADE 330 CS and POLYSURF 67 CS (INCI: Cetyl Hydroxyethylcellulose) by the company Aqualon/Hercules.

The at least one nonionic derivative of cellulose comprising at least one hydrophobic substituent (A) can be present in the compositions according to the present disclosure in an amount ranging from 0.01% to 10% by weight, for example, from 0.05% to 3% by weight, such as from 0.1% to 1% by weight, relative to the total weight of the composition.

The at least one C₈-C₃₀ fatty acid ester (B) that can be used according to the present disclosure is chosen from monoesters and polyesters, for instance, chosen from the monoesters, diesters and triesters derived from the reaction of linear or branched, saturated or unsaturated, optionally hydroxylated monoacids or diacids comprising from 8 to 30 carbon atoms, with saturated or unsaturated, linear, branched or cyclic monoalcohols or polyols comprising from 2 to 1000 carbon atoms and from 1 to 30 hydroxyl group(s).

The fatty acids can be, as a non-limiting example, stearic acid, palmitic acid, lauric acid, oleic acid and myristic acid.

The monoalcohols or polyols can be, as a non-limiting example, ethanol, isopropanol, isooctanol, dodecanol, stearyl alcohol, ethylene glycol, propylene glycol, glycerol, polyethylene glycols, polypropylene glycols, glucose, methyl glucose, sorbitol, sorbitol anhydride and pentaerythritol.

The monoalcohols and polyols which are not polyethylene glycols and/or polypropylene glycols may be optionally polyoxyalkylenated, such as polyoxyethylenated and/or polyoxypropylenated, the number of moles of ethylene oxide and/or of propylene oxide per mole of ester can range from 2 to 400, such as from 2 to 200.

In at least one embodiment, the monoalcohols or polyols, if they are other than polyethylene glycols and/or polypropylene glycols, are not polyoxyalkylenated.

The at least one fatty acid ester that can be used according to the present disclosure can be nonionic, i.e. it does not comprise an ionic charge.

By way of non-limiting example, esters that may be used according to the disclosure, include, isopropyl myristate, stearyl stearate, myristate or palmitate, ethylene glycol monostearate or distearate, polyethylene glycol monostearate or distearate, such as PEG-40 stearate, sorbitan monopalmitate, glyceryl isostearate, propylene glycol dipelargonate, 2-ethylhexyl palmitate, sorbitan tristearate, di(2-ethylhexyl) sebacate, glyceryl trihydroxystearate, cetyl stearate, palmitate or myristate, and myristyl stearate, palmitate or myristate.

In at least one embodiment of the present disclosure, if the alcohol of the ester is a polyol, then this alcohol contains more than 3 carbon atoms.

The at least one C₈-C₃₀ fatty acid ester of the compositions according to the disclosure may be present in an amount ranging from 0.01% to 10% by weight, for instance, from 0.2% to 10% by weight, such as from 0.5% to 6% by weight, relative to the total weight of the composition.

The composition according to the disclosure may also further comprise at least one fatty acid amide. The at least one fatty acid amide that can be used according to the disclosure can be chosen from amides derived from the reaction of an alkanolamine and of a C₁₄-C₃₀ fatty acid. For example, they can be chosen from the amides of a C₂-C₁₀ alkanolamine and of a C₁₄-C₃₀ fatty acid, such as from the amides of a C₂-C₆ alkanolamine and of a C₁₄-C₂₂ fatty acid.

The alkanolamine may be a monoalkanolamine or a dialkanolamine. The fatty acid may be saturated or unsaturated, and linear or branched.

The at least one fatty acid amide that can be used according to the disclosure can be nonionic, i.e. it does not comprise an ionic charge.

The amide of an alkanolamine and of a C₁₄-C₃₀ fatty acid can be chosen from, for example:

oleic acid diethanolamide, such as the amide sold under the trade name MEXANYL® GT by the company Chimex,

myristic acid monoethanolamide, such as the amide sold under the trade name COMPERLAN® MM by the company Cognis,

soybean fatty acid diethanolamide, such as the amide sold under the trade name COMPERLAN® VOD by the company Cognis,

stearic acid ethanolamide, such as the amide sold under the trade name MONAMID® S by the company Uniqema,

oleic acid monoisopropanolamide, such as the amide sold under the trade name WITCAMIDE® 61 by the company Witco,

linoleic acid diethanolamide, such as the amide sold under the trade name PURTON® SFD by the company Zschimmer Schwarz,

stearic acid monoethanolamide, such as the amide sold under the trade name MONAMID® 972 by the company ICI/Uniqema,

behenic acid monoethanolamide, such as the amide sold under the trade name INCROMIDE® BEM by Croda,

isostearic acid monoisopropanolamide, such as the amide sold under the trade name WITCAMIDE® SPA by the company Witco,

erucic acid diethanolamide, such as the amide sold under the trade name ERUCIC ACID DIETHANOLAMIDE by the company Stearineries Dubois,

ricinoleic acid monoethanolamide, such as the amide sold under the trade name RICINOLEIC MONOETHANOLAMIDE by the company Stearineries Dubois.

The at least one fatty acid amide of the compositions according to the disclosure can be present in an amount ranging from 0 to 10%, for example, from 0.2% to 10% by weight, and such as from 0.5% to 6% by weight, relative to the total weight of the composition.

According to at least one embodiment, the dye compositions of the disclosure comprise, in a medium suitable for dyeing:

(A) at least one nonionic derivative of cellulose comprising at least one hydrophobic substituent containing from 8 to 30 carbon atoms as defined above;

(B) (i) at least one amide of an alkanolamine and of a C₁₄-C₃₀ fatty acid, and
(ii) at least one C₈-C₃₀ fatty acid ester; and

(C) at least one oxidation dye, for example, at least one oxidation base and at least one oxidation coupler.

The at least one oxidation dye (C) that can be used according to the disclosure can be chosen from oxidation bases, oxidation couplers, and addition salts thereof.

By way of non-limiting example, the oxidation bases can be chosen from para-phenylenediamines, bisphenylalkylenediamines, para-aminophenols, bis-para-aminophenols, ortho-aminophenols, heterocyclic bases, and addition salts thereof.

Among the para-phenylenediamines, mention may be made, by way of non-limiting example, of para-phenylenediamine, para-toluoylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylene-diamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the acid addition salts thereof.

Among the para-phenylenediamines mentioned above, further non-limiting examples include, para-phenylenediamine, para-toluoylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the acid addition salts thereof.

Among the bisphenylalkylenediamines, mention may, by way of non-limiting example, be made of N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N-bis(4-aminophenyl)tetramethylenediamine, N,N′-bis(β-hydroxyethyl)-N,N′-bis(4-amino-phenyl)tetramethylenediamine, N,N′-bis-(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the acid addition salts thereof.

Among the para-aminophenols, non-limiting mention may be made of, by way of non-limiting example, para-aminophenol, 4-amino-3-methylphenol, 4-amino-3-fluoro-phenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the acid addition salts thereof.

Among the ortho-aminophenols, mention may, by way of non-limiting example, be made of 2-aminophenol, 2-amino-5-methylphenol, 2-amino-6-methylphenol and 5-acetamido-2-aminophenol, and the acid addition salts thereof.

Among the heterocyclic bases, mention may be made, by way of non-limiting example, of pyridine derivatives, pyrimidine derivatives, pyrazole derivatives, pyrazolone derivatives, and addition salts thereof.

Among the pyridine derivatives, non-limiting mention may be made of the compounds described, for example, in British Patent Nos. GB 1 026 978 and GB 1 153 196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine, and the acid addition salts thereof.

Other pyridine oxidation bases that can be used in the present disclosure include, but are not limited to, the 3-aminopyrazolo[1,5-a]pyridine oxidation bases or addition salts thereof described, for example, in French Patent Application No. FR 2801308. By way of example, non-limiting mention may be made of pyrazolo[1,5-a]pyridin-3-ylamine; 2-acetylaminopyrazolo-[1,5-a]pyridin-3-ylamine; 2-morpholin-4-ylpyrazolo[1,5-a]pyridin-3-ylamine; 3-aminopyrazolo[1,5-a]pyridine-2-carboxylic acid; 2-methoxypyrazolo[1,5-a]pyridin-3-ylamino; (3-aminopyrazolo[1,5-a]pyridin-7-yl)methanol; 2-(3-aminopyrazolo[1,5-a]pyridin-5-yl)ethanol; 2-(3-aminopyrazolo[1,5-a]pyridin-7-yl)ethanol; (3-aminopyrazolo[1,5-a]pyridin-2-yl)methanol; 3,6-diaminopyrazolo[1,5-a]pyridine; 3,4-di-aminopyrazolo[1,5-a]pyridine; pyrazolo[1,5-a]pyridine-3,7-diamine; 7-morpholin-4-ylpyrazolo[1,5-a]pyridin-3-ylamine; pyrazolo[1,5-a]pyridine-3,5-diamine; 5-morpholin-4-ylpyrazolo[1,5-a]pyridin-3-ylamine; 2-[(3-aminopyrazolo[1,5-a]pyridin-5-yl)(2-hydroxyethyl)-amino]ethanol; 2-[(3-aminopyrazolo[1,5-a]pyridin-7-yl)(2-hydroxyethyl)amino]ethanol; 3-aminopyrazolo[1,5-a]pyridin-5-ol; 3-aminopyrazolo[1,5-a]pyridin-4-ol; 3-aminopyrazolo[1,5-a]pyridin-6-ol; 3-aminopyrazolo[1,5-a]pyridin-7-ol; and also addition salts thereof with an acid or with a base.

Among the pyrimidine derivatives, non-limiting mention may be made of the compounds described, for example, in German Patent No. DE 2359399; Japanese Patent Nos. JP 88-169571 and JP 05-63124; European Patent No. EP 0 770 375 or International Patent Application Publication No. WO 96/15765, such as 2,4,5,6-tetraminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine, and pyrazolopyrimidine derivatives such as those mentioned in French Patent Application No. FR-A-2 750 048 and among which further non-limiting mention may be made of pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)(2-hydroxyethyl)amino]ethanol, 2-[(7-aminopyrazolo[1,5-a]pyrimidin-3-yl)(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine, 2, 5, N7, N7-tetramethylpyrazolo[1,5-a]pyrimidine-3,7-diamine, 3-amino-5-methyl-7-imidazolylpropylaminopyrazolo[1,5-a]pyrimidine, and addition salts thereof with an acid and tautomeric forms thereof when a tautomeric equilibrium exists.

Among the pyrazole derivatives described herein, non-limiting mention may be made, for example, of the compounds described in German Patent Nos. DE-A-38 43 892 and DE-A-41 33 957 and International Patent Application Publication Nos. WO 94/08969 and WO 94/08970, French Patent Application No. FR-A-2 733 749 and German Patent Application No. DE-A-195 43 988, such as 4,5-diamino-1-methylpyrazole, 4,5-diamino-1-(β-hydroxyethyl)pyrazole, 3,4-diaminopyrazole, 4,5-diamino-1-(4′-chloro-benzyl)pyrazole, 4,5-diamino-1,3-dimethylpyrazole, 4,5-diamino-3-methyl-1-phenyl-pyrazole, 4,5-diamino-1-methyl-3-phenylpyrazole, 4-amino-1,3-dimethyl-5-hydrazino-pyrazole, 1-benzyl-4,5-diamino-3-methylpyrazole, 4,5-diamino-3-tert-butyl-1-methyl-pyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxy-phenyl)pyrazole, le 4,5-diamino-1-ethyl-3-hydroxymethylpyrazole, 4,5-diamino-3-hydroxy-methyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methyl-aminopyrazole and 3,5-diamino-4-(β-hydroxyethyl)amino-1-methylpyrazole, and addition salts thereof.

Among the pyrazolone derivatives that can be used, non-limiting mention may, for example, be made of the following compounds and addition salts thereof:
2,3-diaminodihydropyrazolone;
4,5-diamino-1,2-dimethyl-1,2-dihydropyrazol-3-one;
4-amino-5-methylamino-1,2-dimethyl-1,2-dihydropyrazol-3-one;
4-amino-5-dimethylamino-1,2-dimethyl-1,2-dihydropyrazol-3-one;
4-amino-5-(2-hydroxyethyl)amino-1,2-dimethyl-1,2-dihydropyrazol-3-one;
4-amino-5-(pyrrolidin-1-yl)-1,2-dimethyl-1,2-dihydropyrazol-3-one;
4-amino-5-(piperidin-1-yl)-1,2-dimethyl-1,2-dihydropyrazol-3-one;
4,5-diamino-1,2-di(2-hydroxyethyl)-1,2-dihydropyrazol-3-one;
4-amino-5-methylamino-1,2-di(2-hydroxyethyl)-1,2-dihydropyrazol-3-one;
4-amino-5-dimethylamino-1,2-di(2-hydroxyethyl)-1,2-dihydropyrazol-3-one;
4-amino-5-(2-hydroxyethyl)amino-1,2-di(2-hydroxyethyl)-1,2-dihydropyrazol-3-one;
4-amino-5-(pyrrolidin-1-yl)-1,2-di(2-hydroxyethyl)-1,2-dihydropyrazol-3-one;
4-amino-5-(piperidin-1-yl)-1,2-di(2-hydroxyethyl)-1,2-dihydropyrazol-3-one;
4,5-diamino-1,2-diethyl-1,2-dihydropyrazol-3-one;
4,5-diamino-1,2-diphenyl-1,2-dihydropyrazol-3-one;
4,5-diamino-1-ethyl-2-methyl-1,2-dihydropyrazol-3-one;
4,5-diamino-2-ethyl-1-methyl-1,2-dihydropyrazol-3-one;
4,5-diamino-1-phenyl-2-methyl-1,2-dihydropyrazol-3-one;
4,5-diamino-2-phenyl-1-methyl-1,2-dihydropyrazol-3-one;
4,5-diamino-1-(2-hydroxyethyl)-2-methyl-1,2-dihydropyrazol-3-one;
4,5-diamino-2-(2-hydroxyethyl)-1-methyl-1,2-dihydropyrazol-3-one;
2,3-diamino-6,7-dihydro-1H,5H-pyrazolo[1,2-a]pyrazol-1-one;
2-amino-3-methylamino-6,7-dihydro-1H,5H-pyrazolo[1,2-a]pyrazol-1-one;
2-amino-3-dimethylamino-6,7-dihydro-1H,5H-pyrazolo[1,2-a]pyrazol-1-one;
2-amino-3-ethylamino-6,7-dihydro-1H,5H-pyrazolo[1,2-a]pyrazol-1-one;
2-amino-3-isopropylamino-6,7-dihydro-1H,5H-pyrazolo[1,2-a]pyrazol-1-one;
2-amino-3-(2-hydroxyethyl)amino-6,7-dihydro-1H,5H-pyrazolo[1,2-a]pyrazol-1-one;
2-amino-3-(2-hydroxypropyl)amino-6,7-dihydro-1H,5H-pyrazolo[1,2-a]pyrazol-1-one;
2-amino-3-bis(2-hydroxyethyl)amino-6,7-dihydro-1H,5H-pyrazolo[1,2-a]pyrazol-1-one;
2-amino-3-(pyrrolidin-1-yl)-6,7-dihydro-1H,5H-pyrazolo[1,2-a]pyrazol-1-one;
2-amino-3-(3-hydroxypyrrolidin-1-yl)-6,7-dihydro-1H,5H-pyrazolo[1,2-a]pyrazol-1-one;
2-amino-3-(piperidin-1-yl)-6,7-dihydro-1H,5H-pyrazolo[1,2-a]pyrazol-1-one;
2,3-diamino-6-hydroxy-6,7-dihydro-1H,5H-pyrazolo[1,2-a]pyrazol-1-one;
2,3-diamino-6-methyl-6,7-dihydro-1H,5H-pyrazolo[1,2-a]pyrazol-1-one;
2,3-diamino-6,6-dimethyl-6,7-dihydro-1H,5H-pyrazolo[1,2-a]pyrazol-1-one;
2,3-diamino-5,6,7,8-tetrahydro-1H,6H-pyridazino[1,2-a]pyrazol-1-one;
2,3-diamino-5,8-dihydro-1H,6H-pyridazino[1,2-a]pyrazol-1-one;
4-amino-5-dimethylamino-1,2-diethyl-1,2-dihydropyrazol-3-one;
4-amino-1,2-diethyl-5-ethylamino-1,2-dihydropyrazol-3-one;
4-amino-1,2-diethyl-5-isopropylamino-1,2-dihydropyrazol-3-one;
4-amino-1,2-diethyl-5-(2-hydroxyethylamino)-1,2-dihydropyrazol-3-one;
4-amino-5-(2-dimethylaminoethylamino)-1,2-diethyl-1,2-dihydropyrazol-3-one;
4-amino-5-[bis(2-hydroxyethyl)amino]-1,2-diethyl-1,2-dihydropyrazol-3-one;
4-amino-1,2-diethyl-5-(3-imidazol-1-ylpropylamino)-1,2-dihydropyrazol-3-one;
4-amino-1,2-diethyl-5-(3-hydroxypyrrolidin-1-yl)-1,2-dihydropyrazol-3-one;
4-amino-5-pyrrolidin-1-yl-1,2-diethyl-1,2-dihydropyrazol-3-one;
4-amino-5-(3-dimethylaminopyrrolidin-1-yl)-1,2-diethyl-1,2-dihydropyrazol-3-one; and
4-amino-1,2-diethyl-5-(4-methylpiperazin-1-yl)pyrazolidin-3-one.

The at least one oxidation base may be present in a total amount ranging from 0.001% to 20% by weight, for example, from 0.005% to 10% by weight, such as from 0.1% to 5% by weight, relative to the total weight of the composition.

The at least one oxidation coupler present in the compositions of the disclosure may be chosen from benzene couplers, heterocyclic couplers, naphthalene couplers, and addition salts thereof.

By way of benzene couplers that can be used in the compositions according to the disclosure, non-limiting mention may be made of meta-aminophenols, meta-phenylenediamines, meta-diphenols, and also addition salts thereof.

Among the couplers that may be used as disclosed herein, non-limiting mention may be made of 2-methyl-5-aminophenol, 5-N-(β-hydroxyethyl)amino-2-methylphenol, 6-chloro-2-methyl-5-aminophenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine 3,5-diamino-2,6-dimethoxypyridine, 1-N-(β-hydroxyethyl)amino-3,4-methylenedioxybenzene and 2,6-bis-(β-hydroxyethylamino)toluene, and the acid addition salts thereof.

The at least one oxidation coupler can be present in an amount ranging from 0.001% to 20% by weight, for example, from 0.005% to 10% by weight, such as from 0.01% to 5% by weight, relative to the total weight of the composition.

In general, the addition salts of the oxidation bases and of the couplers that can be used in the context of the disclosure can be chosen from the addition salts with an acid, such as the hydrochlorides, hydrobromides, sulphates, citrates, succinates, tartrates, lactates, tosylates, benzenesulphonates, phosphates and acetates, and the addition salts with a base, such as sodium hydroxide, potassium hydroxide, aqueous ammonia, amines or alkanolamines.

The dye composition described herein may further comprise at least one direct dye that may be chosen from, as non-limiting examples, nitrobenzene dyes, azo direct dyes, methine direct dyes, anthraquinone dyes, xanthene dyes, triarylmethane dyes, and addition salts thereof. These direct dyes may be nonionic, anionic or cationic in nature.

The medium used in the compositions according to the present disclosure can be an aqueous medium or can be a medium comprising water and at least one organic solvent.

The at least one organic solvent used in the compositions according to the disclosure may be chosen from monohydroxylated alcohols and polyols.

By way of monohydroxylated alcohols that can be used, non-limiting mention may be made of C₁-C₄ lower alcohols such as ethanol, isopropanol, tert-butanol, n-butanol, and mixtures thereof. For example, in at least one embodiment, the alcohol is ethanol.

Non-limiting examples of polyols include propylene glycol, polyethylene glycols and glycerol. By way of organic solvents, non-limiting mention may also be made of polyol ethers such as 2-butoxyethanol, propylene glycol monomethyl ether, diethylene glycol monoethyl ether and diethylene glycol monomethyl ether, and also aromatic alcohols such as benzyl alcohol or phenoxyethanol, and mixtures thereof.

The at least one organic solvent in the compositions according to the present disclosure can be present in an amount ranging from 0 to 30%, such as ranging from 0 to 20% by weight, relative to the total weight of the composition.

The compositions according to the present disclosure may further comprise at least one thickener, also referred to as “rheology-adjusting agent,” different from the at least one nonionic derivatives of cellulose with at least one hydrophobic substituent of the disclosure.

In at least one embodiment, the at least one rheology-adjusting agent may be chosen from, but not limited to, mineral or organic thickeners, for instance, polymeric associative thickeners, fatty alcohols (oleyl alcohol), cellulosic derivatives other than the at least one nonionic derivatives of cellulose with the at least one hydrophobic substituent (A) according to the disclosure (hydroxyethylcellulose, hydroxypropylcellulose, carboxymethylcellulose) and gums of microbial origin (xanthan gum, scleroglucan gum).

The at least one rheology-adjusting agent can be chosen from fatty alcohols, such as C₂₀-C₂₂ fatty alcohols, and cellulose derivatives, other than the at least one nonionic derivative of cellulose with at least one hydrophobic substituent (A) according to the disclosure.

The at least one thickener may be present in an amount ranging from 0.01% to 20% by weight, such as ranging from 1% to 10% by weight, relative to the total weight of the composition.

The dye composition described herein may further comprise at least one adjuvant conventionally used in compositions for dyeing the hair.

As used herein, “adjuvant” means at least one additive different from the abovementioned compounds, such as anionic, cationic, nonionic, amphoteric or zwitterionic surfactants or mixtures thereof; nonionic, amphoteric, zwitterionic, anionic or cationic polymers, other than the nonionic derivatives of cellulose with hydrophobic substituent(s) (A) according to the disclosure, or mixtures of said polymers; penetrating agents; sequestering agents; fragrances; buffers; dispersants; conditioning agents such as, for example, modified or unmodified, volatile or non-volatile silicones; film-forming agents; ceramides other than the fatty acid amides (B) (i) according to the disclosure; preservatives; opacifiers; vitamins; amino acids; oligopeptides; peptides; modified or unmodified, hydrolysed or nonhydrolysed proteins; enzymes; branched or unbranched fatty acids and alcohols; animal, plant or mineral waxes; hydroxylated organic acids; UV screens; antioxidants and free-radical scavengers; antidandruff agents; seborrhoea-regulating agents; calmatives; mineral oils; polyisobutenes and poly(α-olefins); pigments; acids, bases, plasticizers, mineral fillers, pearlescent agents, flakes; antistatic agents and reducing agents.

The at least one adjuvant can be present in an amount, for each adjuvant and for instance, ranging from 0.01% to 40% by weight, and such as ranging from 0.1% to 25% by weight, relative to the total weight of the composition.

Of course, those skilled in the art will take care to select this (or these) possible additional compound(s) in such a way that the beneficial properties intrinsically associated with the oxidation dyeing composition in accordance with the disclosure are not, or not substantially, impaired by the addition(s) envisaged.

The pH of the dye composition in accordance with the disclosure can ranges from 3 to 12, and such as from 5 to 11. It may be adjusted to the desired value via acidifying agent(s) or basifying agent(s) commonly used in the dyeing of keratin fibers or alternatively using conventional buffer system(s).

Among the acidifying agents, mention may be made, by way of non-limiting example, of mineral or organic acids such as hydrochloric acid, orthophosphoric acid, sulphuric acid, sulphonic acids and carboxylic acids, for instance acetic acid, tartaric acid, citric acid and lactic acid.

Among the basifying agents, mention may be made, by way of non-limiting example, of aqueous ammonia, alkali metal carbonates, alkanolamines such as mono-, di- and triethanolamines and derivatives thereof, sodium hydroxide or potassium hydroxide and the compounds of formula (I) below:

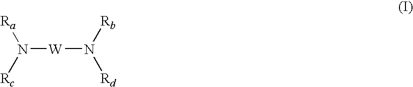

wherein:
W is a propylene residue optionally substituted with a hydroxyl group or a C₁-C₄ alkyl group;
Rₐ, R_{b}, R_{c} and R_{d}, which may be identical or different, are independently chosen from hydrogen atoms, C₁-C₄ alkyl groups, and C₁-C₄ hydroxyalkyl groups.

The dye composition according to the disclosure may be in various forms, such as in the form of creams or gels, or in any other form suitable for dyeing keratin fibers, such as human hair.

The process for dyeing keratin fibers, of the present disclosure, is a process wherein the composition according to the present disclosure as defined above is applied to the fibers, typically in the presence of at least one oxidizing agent for a period of time sufficient to develop the desired color. The color may be revealed at acidic, neutral or alkaline pH and the at least one oxidizing agent may be added to the composition of the disclosure just at the time of use, or it may be used starting from an oxidizing composition comprising it, applied simultaneously with or sequentially to the composition of the disclosure.

According to at least one embodiment, the composition according to the present disclosure is a ready-to-use composition, wherein the dye composition is mixed, for instance, at the time of use, with a composition comprising, in a medium suitable for dyeing, at least one oxidizing agent, the at least one oxidizing agent being present in a sufficient amount to develop a coloration. The mixture obtained is subsequently applied to the keratin fibers. After a period of leave-on time ranging from 3 to 50 minutes, such as from 5 to 30 minutes, the keratin fibers are rinsed, washed with shampoo, rinsed again, and then dried.

The at least one oxidizing agent conventionally used for the oxidation dyeing of keratin fibers can be chosen from, by way of non-limiting example, hydrogen peroxide, urea peroxide, alkali metal bromates, persalts such as perborates and persulphates, peracids and oxidase enzymes, among which non-limiting mention may be made of peroxidases, 2-electron oxidoreductases, such as uricases, and 4-electron oxygenases, such as laccases, these oxidoreductases being optionally combined with their customary cofactors, such as uric acid for uricases. In at least one embodiment, the at least one oxidizing agent is hydrogen peroxide.

The oxidizing composition may also contain at least one adjuvant conventionally used in compositions for dyeing the hair, as defined above.

The pH of the oxidizing composition comprising the at least one oxidizing agent is such that, after mixing with the dye composition, the pH of the resulting composition applied to the keratin fibers can range from 3 to 12, such as from 5 to 10. It may be adjusted to the desired value via acidifying agent(s) or basifying agent(s) normally used in the dyeing of keratin fibers, as defined above.

The ready-to-use composition which is finally applied to the keratin fibers may be in various forms, such as in the form of creams or gels, or in any other form suitable for dyeing keratin fibers, for example, human keratin fibers such as the hair.

Another aspect of the disclosure is a multicompartment dyeing device or dyeing “kit,” comprising at least one first compartment containing the at least one dye composition as described herein, and at least one second compartment containing at least one oxidizing composition. This device may be equipped with a mechanism for delivering the desired mixture to the hair, such as the devices described in French Patent Application FR-A-2 586 913.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

The following examples serve to illustrate embodiments of the present disclosure without, however, exhibiting being a limiting nature.

### EXAMPLES

### Example 1

### Dye Compositions According to the Disclosure

The following Compositions 1 and 2 were prepared.

| | Dye Composition | |
|---|---|---|
| | Compo- | Compo- |
| | sition 1 | sition 2 |
| Cetyl hydroxyethylcellulose | — | 0.5 g |
| (POLYSURF 67 sold by the company | | |
| Aqualon) | | |
| Cetyl hydroxyethylcellulose | 0.4 g | — |
| (NATROSOL PLUS GRADE 330 CS sold by | | |
| the company Aqualon) | | |
| Stearic acid monoethanolamide | 4.8 g | 4.8 g |
| PEG-40 stearate | — | 1.8 g |
| p-aminophenol | — | 0.24 g |
| Toluene-2,5-diamine | 0.76 g | 0.28 g |
| Resorcinol | 0.69 g | — |
| m-aminophenol | 0.14 g | 0.02 g |
| 2,4-diaminophenoxyethanol, HCl | 0.02 g | 0.02 g |
| 2-amino-3-hydroxypyridine | — | 0.2 g |
| 6-hydroxyindole | — | 0.01 g |
| 4-amino-2-hydroxytoluene | — | 0.24 g |
| 2-methyl-5-hydroxyethylaminophenol | — | 0.15 g |
| Oleic acid | 3 g | 3 g |
| Aqueous solution containing 20% by weight | 5 g | 5 g |
| of NH₃ | | |
| TiO₂ | 0.3 g | 0.3 g |
| Monoethanolamine | 0.8 g | 0.8 g |
| Oleth-10 | 1.8 g | — |
| Aqueous solution containing 40% by weight | 1.6 g | 1.6 g |
| of Polyquaternium-6 | | |
| (MERQUAT 100 sold by the company Ondeo) | | |
| Ethylenediaminetetraacetic acid (EDTA) | 0.2 g | 0.2 g |
| Aqueous solution of 60% by weight | 1.2 g | 1.2 g |
| of hexadimethrine chloride | | |
| (MEXOMERE PO sold by the company | | |
| Chimex) | | |
| Hydroxypropylmethylcellulose | 0.19 g | 0.19 g |
| Oleth-30 | 1.5 g | 1.5 g |
| Steareth-2 | 5.5 g | 5.5 g |
| Glyceryl lauryl ether | — | 0.5 g |
| C₂₀-C₂₂ alcohols | 3 g | 3 g |
| (NAFOL 2022 EN sold by the | | |
| company Sasol) | | |
| Reducing agent, antioxidant | q.s. | q.s. |
| Demineralized water q.s. | 100 g | 100 g |

### Application Protocol

Both Composition 1 and 2 were each separately diluted, extemporaneously, with one and a half times its weight of an oxidizing composition having a pH in the region of 3 (aqueous hydrogen peroxide at 20 volumes) (6% by weight of H₂O₂). The mixtures were easily prepared and had a good viscosity; they were easily applied to grey hair, comprising 90% white hairs, at a rate of 10 g per 1 g of hair, and left on for 30 minutes. The hair was then rinsed, washed with a standard shampoo and dried.

The hair coloration was evaluated visually. The results obtained on natural grey hair, comprising 90% white hairs, after treatment, were the following:

| | Shade |
|---|---|
| Composition 1 | Mahogany red chestnut |
| Composition 2 | Coppery mahogany blond |

These colorations had good properties, in particular in terms of selectivity and strength. The compositions obtained were stable over time.

### Example 2

### Comparative Testing

Composition 3 according to the disclosure and comparative Composition 4 were prepared.

| | Dye composition | | | |
|---|---|---|---|---|
| | Composition 3 | | Composition 4 | |
| | (inventive) | | (comparative) | |
| Ammonium hydroxide | 10 | g | 10 | g |
| Erythorbic acid | 0.50 | g | 0.50 | g |
| Ethanolamine | 0.70 | g | 0.70 | g |
| EDTA | 0.20 | g | 0.20 | g |
| Sodium sulphite | 0.50 | g | 0.50 | g |
| Titanium dioxide | 0.25 | g | 0.25 | g |
| 4-Amino-2-hydroxytoluene | 0.22 | g | 0.22 | g |
| p-Phenylenediamine | 3 | g | 3 | g |
| Cetearyl alcohol | 0.45 | g | 0.45 | g |
| Myristyl lactate | — | | 0.67 | g |
| | | | (0.00235 | mol) |
| Myristyl myristate | 1 | g | — | |
| | (0.00235 | mol) | | |
| Oleic acid | 3 | g | 3 | g |
| Oxyethylenated stearyl alcohol | 5.50 | g | 5.50 | g |
| comprising 2 mol of ethylene oxide | | | | |
| Oxyethylenated oleocetyl alcohol | 1.50 | g | 1.50 | g |
| comprising 30 mol of ethylene | | | | |
| oxide | | | | |
| Water | qs 100 | g | qs 100 | g |

### Application Protocol

At the time of use, each of Compositions 3 and 4 was mixed separately with one and a half times its weight of an oxidizing composition (aqueous hydrogen peroxide at 20 volumes) (6% by weight of H₂O₂).

### Dyeing Properties

Each mixture was applied to natural (NW) and permanent-waved (PW) locks of hair containing 90% white hairs, at a rate of 15 g of mixture per gram of locks of hair. After a leave-on time of 30 minutes at ambient temperature, the locks were rinsed, washed with a standard shampoo, rinsed again and dried.

The clorimetric measurements were carried out using the Konica Minolta CM-2600d spectrocolorimeter in the CIE L*a*b* system. In the L*a*b* system, L* represents the strength of the coloring obtained; the lower the value of L*, the stronger the coloring obtained. The chromaticity is measured by the values a* and b*, a* indicating the value along the green/red color axis and b* indicating the value along the blue/yellow color axis.

For each composition, the selectivity of the coloring was evaluated. The selectivity of the coloring was the variation in the color between natural hair and permanent-waved hair. The natural hair was representative of the nature of the hair at the root, whereas the permanent-waved hair was representative of the nature of the hair at the end.

The selectivity was measured by ΔE, which is the variation in color between the natural hair and the permanent-waved hair, and is obtained from the formula:

ΔE=\sqrt{(L*−Lₒ*)²+(a*−aₒ*)²+(b*−bₒ*)²}

in which:

L*, a* and b* represent the parameters of the dyed permanent-waved hair, and

Lₒ*, aₒ* and bₒ* represent the parameters of the dyed natural hair.

The lower the value of ΔE, the lower the selectivity and therefore the more uniform the coloring along the hair.

### Results

| | Hair type | L* | a* | b* | ΔE |
|---|---|---|---|---|---|
| Composition 3 | NW dyed | 24.68 | 15.49 | 0 | 3.60 |
| | PW dyed | 21.24 | 15.37 | −1.04 | |
| Composition 4 | NW dyed | 23.58 | 16.59 | −0.57 | 6.15 |
| | PW dyed | 18.98 | 12.51 | −0.33 | |

The selectivity (ΔE) was lower for Composition 3 according to the present disclosure, thereby demonstrating a more uniform coloring along the fiber.

What is claimed is:

1. A composition for dyeing keratin fibers, comprising, in a medium suitable for dyeing:
(A) at least one nonionic derivative of cellulose comprising at least one hydrophobic substituent containing from 8 to 30 carbon atoms;
(B) at least one C₈-C₃₀ fatty acid ester; and
(C) at least one oxidation dye.

2. The composition according to claim 1, wherein the at least one nonionic derivative of cellulose is chosen from a hydroxyethylcellulose substituted with at least one hydrophobic substituent containing from 8 to 30 carbon atoms.

3. The composition according to claim 1, wherein the at least one hydrophobic substituent is chosen from C₁₀-C₂₂ alkyl groups.

4. The composition according to claim 3, wherein the at least one hydrophobic substituent is chosen from cetyl groups.

5. The composition according to claim 1, wherein the degree of hydrophobic substitution ranges from 0.1% to 10% by weight of the total weight of the polymer.

6. The composition according to claim 5, wherein the degree of hydrophobic substitution ranges from 0.1% to 1% by weight of the total weight of the polymer.

7. The composition according to claim 6, wherein the degree of hydrophobic substitution ranges from 0.4% to 0.8% by weight of the total weight of the polymer.

8. The composition according to claim 1, wherein the at least one nonionic derivative of cellulose is present in an amount ranging from 0.01% to 10% by weight, relative to the total weight of the composition.

9. The composition according to claim 8, wherein the at least one nonionic derivative of cellulose is present in an amount ranging from 0.05% to 3% by weight, relative to the total weight of the composition.

10. The composition according to claim 9, wherein the at least one nonionic derivative of cellulose is present in an amount ranging from 0.1% to 1% by weight, relative to the total weight of the composition.

11. The composition according to claim 1, further comprising at least one fatty acid amide chosen from those of an alkanolamine and of a C₁₄-C₃₀ fatty acid.

12. The composition according to claim 11, wherein the at least one fatty acid amide is chosen from those of a C₂-C₁₀ alkanolamine and of a C₁₄-C₃₀ fatty acid.

13. The composition according to claim 12, wherein the at least one fatty acid amide is chosen from those of a C₂-C₆ alkanolamine and of a C₁₄-C₂₂ fatty acid.

14. The composition according to claim 11, wherein the at least one alkanolamine is chosen from monoalkanolamine and dialkanolamine.

15. The composition according to claim 11, wherein the at least one fatty acid amide is chosen from:
oleic acid diethanolamide,
myristic acid monoethanolamide,
soybean fatty acid diethanolamide,
stearic acid ethanolamide,
oleic acid monoisopropanolamide,
linoleic acid diethanolamide,
stearic acid monoethanolamide,
behenic acid monoethanolamide,
isostearic acid monoisopropanolamide,
euric acid diethanolamide, and
ricinoleic acid monoethanolamide.

16. The composition according to claim 11, wherein the at least one fatty acid amide is present in an amount ranging from 0.2% to 10% by weight, relative to the total weight of the composition.

17. The composition according to claim 16, wherein the at least one fatty acid amide is present in an amount ranging from 0.5% to 6% by weight, relative to the total weight of the composition.

18. The composition according to claim 1, wherein the at least one C₈-C₃₀ fatty acid ester is chosen from monoesters, diesters and triesters derived from the reaction of linear or branched, saturated or unsaturated, optionally hydroxylated monoacids or diacids containing from 8 to 30 carbon atoms, with saturated or unsaturated, linear, branched or cyclic monoalcohols or polyols comprising from 2 to 100 carbon atoms and from 1 to 30 hydroxyl group(s).

19. The composition according to claim 1, wherein the at least one C₈-C₃₀ fatty acid ester is chosen from isopropyl myristate; stearyl stearate, myristate and palmitate; ethylene glycol monostearate and distearate; polyethylene glycol monostearate and distearate; sorbitan monopalmitate; glyceryl isostearate; propylene glycol dipelargonate; 2-ethylhexyl palmitate; sorbitan tristearate; di(2-ethylhexyl)sebacate; glyceryl trihydroxystearate; cetyl stearate, palmitate and myristate; and myristyl stearate, palmitate and myristate.

20. The composition according to claim 1, wherein the at least one C₈-C₃₀ fatty acid ester is present in an amount ranging from 0.01% to 10% by weight, relative to the total weight of the composition.

21. The composition according to claim 20, wherein the at least one C₈-C₃₀ fatty acid ester is present in an amount ranging from 0.2% to 10% by weight, relative to the total weight of the composition.

22. The composition according to claim 21, wherein the at least one C₈-C₃₀ fatty acid ester is present in an amount ranging from 0.5% to 6% by weight, relative to the total weight of the composition.

23. The composition according to claim 1, wherein the at least one oxidation dye is chosen from oxidation bases, oxidation couplers, and addition salts thereof.

24. The composition according to claim 23, wherein the at least one oxidation base is chosen from para-phenylenediamines, bisphenylalkylenediamines, para-aminophenols, bis-para-aminophenols, ortho-aminophenols, heterocyclic bases, and addition salts thereof.

25. The composition according to claim 23, wherein the at least one oxidation base is present in a total amount ranging from 0.001% to 20% by weight, relative to the total weight of the composition.

26. The composition according to claim 25, wherein the at least one oxidation base is present in an amount ranging from 0.005% to 10% by weight, relative to the total weight of the composition.

27. The composition according to claim 26, wherein the at least one oxidation base is present in an amount ranging from 0.01% to 5% by weight, relative to the total weight of the composition.

28. The composition according to claim 23, wherein the at least one oxidation coupler is chosen from benzene couplers, heterocyclic couplers, naphthalene couplers, and addition salts thereof.

29. The composition according to claim 28, wherein the at least one benzene coupler is chosen from meta-aminophenols, meta-phenylenediamines, meta-diphenols, and addition salts thereof.

30. The composition according to claim 23, wherein the at least one oxidation coupler is present in an amount ranging from 0.001% to 20% by weight, relative to the total weight of the composition.

31. The composition according to claim 30, wherein the at least one oxidation coupler is present in an amount ranging from 0.005% to 10% by weight, relative to the total weight of the composition.

32. The composition according to claim 30, wherein the at least one oxidation coupler is present in an amount ranging from 0.01% to 5% by weight, relative to the total weight of the composition.

33. The composition according to claim 1, further comprising at least one direct dye chosen from nitrobenzene dyes, azo direct dyes, methine direct dyes, anthraquinone dyes, xanthene dyes, triarylmethane dyes, and addition salts thereof.

34. The composition according to claim 1, further comprising at least one oxidizing agent.

35. A process for the oxidation dyeing of keratin fibers, comprising applying to the fibers, in the presence of at least one oxidizing agent, for a period of time sufficient to develop the desired color, a dye composition comprising, in a medium suitable for dyeing:
(A) at least one nonionic derivative of cellulose comprising at least one hydrophobic substituent containing from 8 to 30 carbon atoms;
(B) at least one C₈-C₃₀ fatty acid ester; and
(C) at least one oxidation dye

36. A multicompartment device comprising,
at least one first compartment comprising a dye composition comprising, in a medium suitable for dyeing:
(A) at least one nonionic derivative of cellulose comprising at least one hydrophobic substituent containing from 8 to 30 carbon atoms;
(B) at least one C₈-C₃₀ fatty acid ester; and
(C) at least one oxidation dye; and
at least one second compartment comprising at least one oxidizing agent.
